(19) United States
(12) Patent Application Publication
(10) Pub. No.: US 2024/0000516 A1
(43) Pub. Date: Jan. 4, 2024

(54) System for Localization of Anatomical Sites

(71) Applicant: The Regents of the University of California, Oakland, CA (US)

(72) Inventor: Marc-Fernand Ralph Erian, San Francisco, CA (US)

(21) Appl. No.: 17/926,921

(22) PCT Filed: Jun. 9, 2021
(86) PCT No.: PCT/US2021/036604

§ 371 Date: Nov. 21, 2022

Related U.S. Application Data
(60) Provisional application No. 63/046,130, filed on Jun. 30, 2020.
(60) Provisional application No. 63/039,698, filed on Jun. 16, 2020.

(51) Int. Cl. A61B 34/20; A61B 90/00; A61B 10/02; A61B 5/055; A61B 5/00

(52) U.S. Cl. CPC A61B 34/20; A61B 90/39; A61B 10/0233; A61B 5/055; A61B 5/004; A61B 90/37; A61B 2090/3945; A61B 2090/3991; A61B 2034/2065; A61B 2090/374

(57) ABSTRACT

The disclosure pertains to a system for localizing an anatomical site of interest. The system comprises a grid localization system (GLS) comprising a plurality of access regions and one or more indicators configured to identify from the plurality of access regions an access region of interest for accessing the anatomical site of interest. The GLS can also comprise a depth indicator. The system can further comprise, in communication with the GLS, a processor and a computer-readable medium comprising instructions that, when executed by the processor, causes the one or more indicators to identify the access region of interest. Furthermore, the system can comprise an imaging unit. Even further, the system can comprise an automated access device that accesses the anatomical site of interest based on input from the GLS and/or the processor. Also provided are methods of accessing an anatomical site of interest using the systems disclosed herein.

### CROSS-REFERENCE

This application claims priority to U.S. Provisional Application 63/039,698, filed Jun. 16, 2020, and U.S. Provisional Application 63/046,130, filed on Jun. 30, 2020 which is incorporated herein by reference.

### INTRODUCTION

MRI-guided biopsy procedures are routinely used to obtain biopsies. Under the conventional protocols, a patient is positioned, intravenous contrast medium is administered, and dynamic post-contrast images of the anatomy of interest are acquired. The lesion is then targeted using a vendor software in the MRI-control room. In the case of MRI-breast biopsies, the location of the target lesion, including the grid coordinate, plug location, and lesion depth are manually written on a paper, which is then physically taken into the MRI suite and is placed next to the grid to guide the proceduralist. The proceduralist manually counts the grid spaces to confirm the correct grid location. This process requires time and often requires help from the nurse/technologist. Therefore, this process is slower and prone to human error.

### SUMMARY

Certain embodiments of the invention provide devices and methods that automate the transcription process for identifying the location of an anatomical site of interest, such as a biopsy lesion, in an imaging-guided procedure.

The system disclosed herein for localizing an anatomical site of interest in a subject, comprises a grid localization system (GLS), the GLS comprising a plurality of access regions for accessing the subject and one or more indicators configured to identify from the plurality of access regions an access region of interest for accessing the anatomical site of interest. The GLD can be further configured to indicate, for example, through a digital screen, the depth of the anatomical site of interest.

The system disclosed herein can further comprise a processor in communication with the GLD and a computer-readable medium comprising instructions that, when executed by the processor, causes the one or more indicators to identify from the plurality of access regions the access region of interest for accessing the anatomical site of interest. The system can also comprise an imaging unit configured to image the subject.

Certain embodiments of the invention also provide an automated access device (AAD) that is in communication with the GLS and/or the processor, wherein the AAD, when actuated, accesses the anatomical site of interest in the subject based on the access region of interest and/or the depth of the anatomical site of interest as identified by the GLS and/or the processor.

Further embodiments of the invention provide methods of accessing an anatomical site of interest in a subject by placing the GLS disclosed herein at or near the anatomical site of interest and accessing the anatomical site of interest based on the access region of interest and/or the depth of the anatomical site of interest as identified by the indicator. Such accessing the anatomical site can comprise obtaining a biopsy sample, such as breast or prostate biopsy sample, from the anatomical site or injecting a compound at the anatomical site of interest.

### BRIEF DESCRIPTION OF THE FIGURES

FIG. 1: An example of the system comprising GLS and other components. The illustration of the breast biopsy apparatus is based on commercially available “Breast Biopsy 7-Channel Coil BI 7” by NORAS™ MRI products.

FIG. 2: Schematic of an example of MRI-Biopsy Grid Localization System (MGLS) add-on user interface and transmission/receiver system. The illustration of the work-station screen is based on commercially available breast MRI analysis system DynaCAD Breast™.

FIG. 3: Schematic of an example of MGLS with integrated user interface and transmission/receiver system. The illustration of the work-station screen is based on commercially available breast MRI analysis system DynaCAD Breast™.

FIG. 4: An example of an activated grid during procedure demonstrating illuminated lighting elements and an example of activated GLS during procedure demonstrating display module.

FIG. 5: An example of region of interest (ROI) query tool. The illustration of the work-station screen is based on commercially available breast MRI analysis system DynaCAD Breast™.

FIG. 6: An example of the system of the disclosure during medial biopsy with lighting elements.

FIG. 7: An example of MGLS with an automated biopsy device (ABD).

FIG. 8: An example of a grid rail system for ABD showing a wiring scheme for MGLS.

FIG. 9: An example of a GLS having LED light indicators and digital depth indicator. In this example, the GLS comprises a plastic open-grid which can be affixed to existing MRI breast biopsy grid system. The GLS comprises a matrix of illuminated Cartesian coordinates (XY) and display module to display Z coordinate/depth. The GLS can also have an MRI-compatible Bluetooth receiver module. A separate Bluetooth transmission device with user interface can be provided to communicate with this GLS. Alternatively, the Bluetooth transmission device can be integrated into existing commercially available targeting software systems, for example, Phillips DynaCAD™, Hologic Aegis™, and Sectra Medical™.

FIG. 10: An example of localization of an anatomical site of interest in breast biopsy. Once an anatomical site of interest, i.e., a breast lesion for biopsy, has been selected utilizing a processor, the location of the anatomical site of interest in terms of access region of interest and depth is transmitted via Bluetooth signal to the GLS. The GLS then illuminates the access region of interest for the biopsy and provides a digitized readout of the depth of the lesion to the proceduralist.

FIG. 11: An example of the transmission of the targeting data from the processor the GLS. A proceduralist can target an anatomical site of interest and cause the processor to transmit the coordinates of the anatomical site of interest to a portable targeting key, which is a portable electronic device configured to store, process, and transmit data. The portable targeting key can be transported to the GLS. The targeting key can then be connected to the GLS via a wired or wireless connection. The targeting key can then provide the coordinates of the anatomical site of interest to the GLS and illuminate the appropriate lighting elements for the access area of interest. The targeting key display could also be used in lieu of a display on the actual biopsy on the grid.

### DETAILED DESCRIPTION

Magnetic resonance imaging (MRI) guided-biopsy is an important tool in the armamentarium for cancer diagnosis utilizing minimally invasive tissue sampling and lesion localization. MRI imaging provides the primary advantage of increased sensitivity for detecting malignant lesions which may be occult utilizing conventional imaging modalities. The increased adoption of MR-imaging for the detection of numerous cancers, including cancers of the breast and prostate, have generated a concomitant increase in the number of MRI-guided biopsies performed globally. Utilization of an MRI-compatible localization device coupled with a grid system is the standard method utilized for MRI-guided biopsies in conventional methods.

The GLS disclosed herein can aid the proceduralist by eliminating the need for manual transcription of the coordinates of an anatomical site of interest, such as a biopsy site, and the manual confirmation of the correct grid coordinate at the time of a procedure. GLS indicates the location for an anatomical site of interest, for example, via illuminating on the GLS the location of the anatomical site of interest. The location of the anatomical site of interest can be communicated to the GLS following anatomical site imaging and targeting on a vendor software or following biopsy data entered by an operator.

The devices and methods disclosed herein allow for process simplification by coupling anatomical site targeting at the workstation with anatomical site localization on the grid, facilitating timely and accurate transmission of data. Direct data transmission between the workstation and the GLS placed at or near the subject reduces human error and the likelihood of wrong site sampling and reduces the need for repeat procedures.

In case of MRI-guided biopsies, the reduced procedural time allows for less time for contrast washout and a larger time window for additional sampling if needed following the initial biopsy. Decreased overall procedure time allows for a valuable decrease in the utilization of the MRI scanner and increased time available for diagnostic imaging. These benefits would be compounded when utilized for multiple-site breast biopsies, when time is a critical for successful biopsy execution and when the risk of human error is the highest.

Accordingly, in certain embodiments, the disclosure pertains to devices and methods that allow for improved speed and accuracy of imaging-guided procedures, such as imaging-guided biopsies. The devices and methods disclosed herein provide benefits over the conventional devices and methods, such as improved pre-procedural targeting, reduced human error, and improved accuracy.

Before the methods, computer-readable media and devices of the present disclosure are described in greater detail, it is to be understood that the methods, computer-readable media, and devices are not limited to the embodiments described, as such may, of course, vary. It is also to be understood that the terminology used herein is for the purpose of describing the embodiments only, and is not intended to be limiting, since the scope of the methods, computer-readable media, and devices will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges and are also encompassed, subject to any specifically excluded limit in the stated range. Where the stated range includes one or both the limits, ranges excluding either or both of those included limits are also included.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, computer-readable media, and devices belong. Although any methods, computer-readable media and devices similar or equivalent to those described herein can also be used in the practice or testing of the methods, computer-readable media and devices, representative illustrative methods, computer-readable media and devices are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, computer-readable media and devices are not entitled to antedate such publication, as the date of publication provided may be different from the actual publication date which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements or use of a “negative” limitation.

It is appreciated that certain features of the methods, computer-readable media and devices, which are, for clarity, described in the context of separate embodiments, may also be provided in combination in a single embodiment. Conversely, various features of the methods, computer-readable media and devices, which are, for brevity, described in the context of a single embodiment, may also be provided separately or in any suitable sub-combination. All combinations of the embodiments are specifically embraced by the present disclosure and are disclosed herein just as if each combination was individually and explicitly disclosed, to the extent that such combinations embrace operable processes and/or compositions. In addition, all sub-combinations listed in the embodiments describing such variables are also specifically embraced by the present methods, computer-readable media and devices and are disclosed herein just as if each such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods, computer-readable media, and devices. Any recited method can be carried out in the order of events recited or in any other order that is logically possible.

### Devices

Certain embodiments of the invention disclose a system for localizing an anatomical site of interest in a subject, comprising a grid localization system (GLS). The GLS comprises a plurality of access regions for accessing the subject and one or more indicators configured to identify from the plurality of access regions an access region of interest for accessing the anatomical site of interest.

An “anatomical site” refers to a region within the anatomy of a subject. When used in the context of an “anatomical site,” the term “localize,” “localizing,” or grammatical variations thereof refer to identifying the location of the anatomical site within the body of a subject.

The phrase “anatomical site of interest” refers to a region within the anatomy of a subject that concerns prevention, diagnosis, or treatment of a disease. For example, an anatomical site of interest can be a potentially cancerous lesion in the tissue of a subject and a sample of the tissue from the anatomical site of interest could be used to diagnose cancer in the subject. An anatomical site of interest can also be a potentially cancerous lesion in the tissue of a subject and removing or destroying the tissue from the anatomical site of interest could be used to prevent the potential spread of cancer in the subject. An anatomical site of interest can also be a cancerous lesion in the tissue of a subject and an anti-cancer therapeutic can be specifically administered to the anatomical site of interest to treat the cancer.

Of course, cancer is used herein as an exemplary disease and an anatomical site of interest could be associated with any disease for which a preventive, diagnostic, or therapeutic treatment can be targeted to an anatomical site of interest.

The GLS comprises a plurality of access regions for accessing the subject. The access regions on the GLS typically comprise holes. The holes allow access to the subject, for example, one can insert a needle through the subject's skin via the access regions on the GLS. The holes also allow a user to view the area of the subject that can be accessed via the access region.

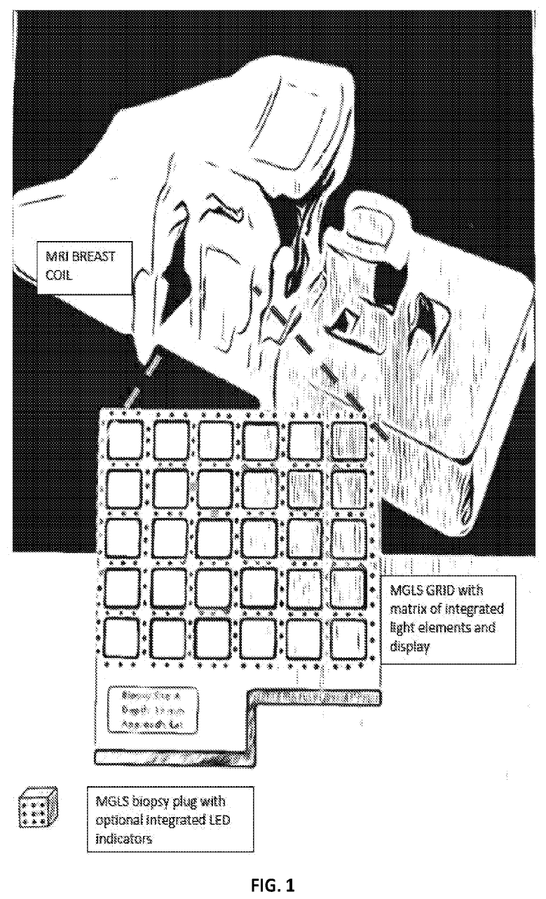

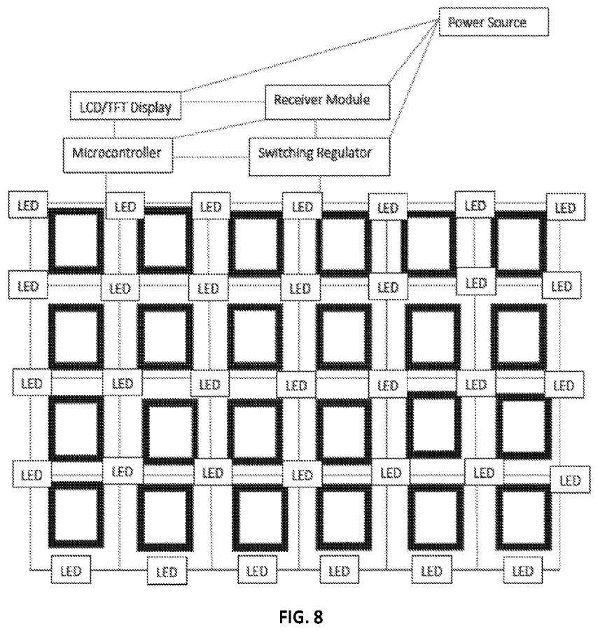

An example of a GLS comprising holes as access regions is provided in FIGS. 1 and 8. In these figures, the GLS comprises a grid comprising a plurality of square access regions in the form of square holes.

While the access regions are typically holes, the access regions can also comprise of material that could be easily pierced, for example, using a sharp tip of a needle. While any suitable material could be used, examples of material that could be easily pierced by a needle includes flexible paper or soft plastic. When the access regions comprise of a material that could be easily pierced, the material can be transparent or substantially transparent. A transparent or substantially transparent material allows a user to view the area of the subject that can be accessed via the access region.

Typically, a GLS is placed on the skin of a subject that is near the anatomical region that contains or is suspected to contain the anatomical site of interest. For example, when the anatomical site of interest is a potentially cancerous lesion in the breast of a subject, a GLS is placed touching the skin on the breast of the subject. Similarly, when the anatomical site of interest is a potentially cancerous lesion in the liver of a subject, a GLS is placed touching the skin on the abdominal area of the subject near the liver.

The GLS disclosed herein can be used to access anatomical site of interest within any organ or tissue, such as placenta, brain, thyroid, parathyroid, thorax, heart, lung, esophagus, thymus, pleura, adrenal glands, appendix, gall bladder, urinary bladder, large intestine, small intestine, kidneys, liver, pancreas, spleen, stoma, prostate, ovaries, uterus, or testis. As noted above, a GLS can be placed touching the skin of the subject near the organ or tissue of interest. Placing a GLS on a skin can be facilitated by an adhesive so that the GLS sticks to the skin. Therefore, in some cases, the GLS comprises an adhesive, for example, on the side opposite to the side comprising the one or more indicators. When used directly on the skin, the adhesive is a skin compatible adhesive. Any suitable adhesive that is compatible with the use on a skin can be used.

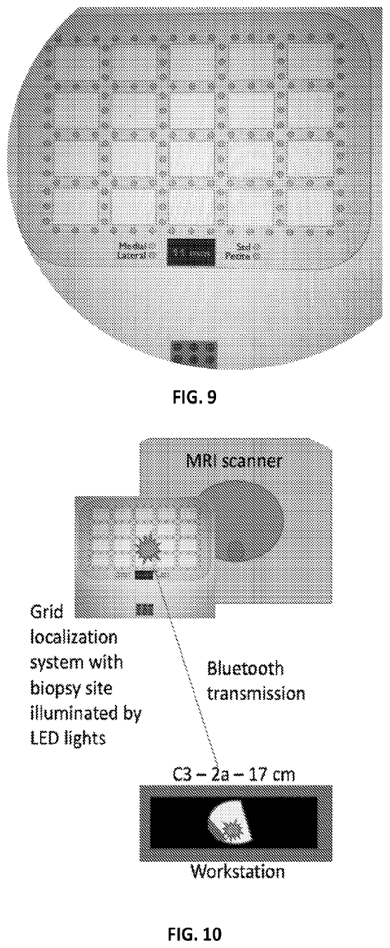

In some cases, the GLS comprises a plurality of quadrangular access regions wherein the one or more indicators are placed along the sides of the quadrangular access regions. The quadrangular access regions could be square or rectangular access regions. Certain such examples are show in FIGS. 9-10. Any suitable shape, such as triangle, pentagonal, hexagonal, oval, elliptical, or circular could be used for preparing an access region of interest. The one or more indicators could be placed along the edges of the access regions.

The one or more indicators of the GLS identify the access region of interest. Typically, the one or more indicators are located along the edges of the plurality of access regions. In some cases, the indicators comprise visual indicators that visually indicate the access region of interest. The visual indicators can be light elements, such as light-emitting diode that can be illuminated around the access region of interest thereby identifying the access region of interest. Alternatively, visual indicators only around the access region of interest can be turned off thereby indicating the access region of interest.

The access region of interest to be used in a particular procedure can determined by an operator or a processor based on the imaging data of the anatomy of the subject. The transmission of information to the GLS about the access region of region of interest determined by an operator or a processor is discussed below in connection with additional details of the system disclosed herein.

Typically, an access region of interest is the access region within the plurality of access regions that can be most effectively used to access the anatomical site of interest. In certain cases, an access region of interest can be most effectively used to access the anatomical site of interest because it corresponds to the area of the skin that is closest to the anatomical site of interest. In certain cases, an access region of interest can also be most effectively used to access the anatomical site of interest because it corresponds to the area of the skin from which the anatomical site of interest can be accessed by avoiding vital structures within the anatomy of the subject. For example, when accessing the anatomical site of interest via the region of the skin closest to the anatomical site of interest may be hindered by vital structures, the anatomical site of interest is typically accessed from other areas of the skin so that the vital structures are not affected by accessing the anatomical site of interest.

In addition to identifying the access region of interest, the GLS can comprise a depth indicator configured to indicate the depth of the anatomical site of interest.

When GLS communicates with an automated access device (AAD, discussed below), the GLS can indicate the access region of interest and/or the depth to the AAD electronically, for example, via wired or wireless communication. In such cases, the GLS may not contain one or more indicators, particularly, one or more visual indicators. Alternatively, when GLS communicates with an AAD, the GLS can indicate the access region of interest and/or the depth to the AAD magnetically, for example, by magnetizing the edges around an access region of interest, wherein the AAD is configured to identify the magnetized edges around the access region of interest.

As used herein, “depth of the anatomical site of interest” refers to the distance of the anatomical site of interest from the skin surface corresponding to the access region of interest. The depth of the anatomical site of interest dictates the length of the apparatus, such as a needle or obturator, that can be introduced within the subject. For example, in case of a breast procedure, “depth of 11 mm” indicates that the anatomical site of interest is located 11 mm from the skin of the subject and a proceduralist would insert a needle so that the tip of the needle is at 11 mm from the skin of the subject.

Typically, the depth of the apparatus introduced into the subject is controlled by a depth stop of an introducer stylet. For example, an operator can adjust the depth stop on the introducer sheath that covers an introducer stylet so that the apparatus introduced through the introducer stylet reaches at the desired depth and, hence, at the anatomical site of interest.

The depth indicator on the GLS can be a screen configured to indicate the depth of the anatomical site of interest. For example, a depth indicator can be an LED display that can display the depth of the anatomical site of interest, such as the LED display depicted in FIG. 9. The depth indicator can also depict a ruler with a mark indicating the depth of the anatomical site of interest. Any other suitable means could be used to indicate the depth of the anatomical site of interest.

In certain embodiments, the system for localizing an anatomical site of interest further comprises, in communication with the GLS, a processor and a computer-readable medium comprising instructions that, when executed by the processor, causes the one or more indicators to identify from the plurality of access regions the access region of interest for accessing the anatomical site of interest.

An operator can use the processor and the computer readable medium to transmit to the GLS the information about the access region of interest and/or depth of the anatomical site of interest. Such transmission can be performed via a wired connection or a wireless connection between the GLS and the processor.

A wireless connection between the GLS and the processor can be Bluetooth, Wi-Fi, Wi-MAX, or cellular connection. A cellular connection can be 3G, 4G, LTE, or 5G connection. Any other suitable connection could also be used to transmit information from the processor to the GLS.

Bluetooth, Wi-Fi, and Wi-MAX connections are typically local area connections and can be used where the processor is located near the subject, such as in the same clinic. A cellular connection can be used when the subject is located at a site distant from the site where the processor is located. For example, imaging data about the subject can be transferred via a cellular network to a distant site and an operator at the distant site can determine the access region of interest based on the imaging data and transmit the information about the access region of interest via the cellular network to the GLS.

An alternative for the transmission of the data about the anatomical site of interest from the processor to the GLS comprises a portable electronic device, referenced herein as “targeting key.” In such communication, the processor is configured to transmit the information about the anatomical site of interest via the targeting key, which is a portable electronic device configured to store, process, and transmit data. The targeting key can have an integrated display, such as LCD or TFT display, and a battery, preferably, a rechargeable battery. The targeting key can be configured to dock at a charger next to the workstation in the imaging area.

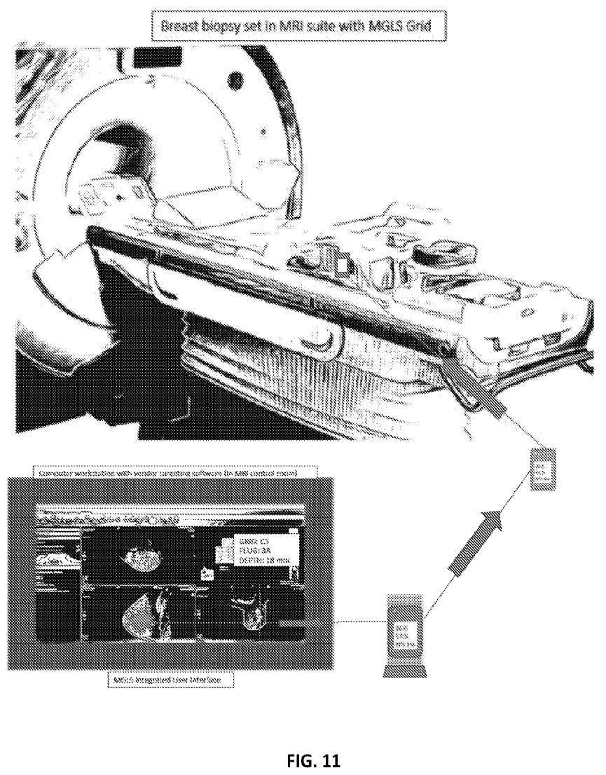

A processor can transmit the information to the targeting key (FIG. 11). A targeting key can communicate with the processor via wired connection, such as USB connection, or wireless connection, such as wirelessly via Bluetooth, Wi-Fi, or Wi-MAX connection.

After identifying the anatomical region of interest, for example, using an imaging unit and processing program, an operator can transmit the information about the access region of interest to the targeting key. If the targeting key has a display, the appropriate coordinates can be displayed on such display.

The targeting key can also be configured to allow for manual input of the relevant coordinates of the anatomical site of interest. This targeting key could then be carried to the site of the procedure. At the site of the procedure, the targeting key can either directly connect to the GLS via a physical connection or could wirelessly transmit the targeting data to the GLS, illuminating the appropriate lighting elements of the access region of interest for accessing the anatomical site of interest.

The targeting key can also be configured to snap onto the GLS or other instruments used in the medical procedure, such as MRI table or MRI breast coil. The display of the targeting key can be configured to display additional data about the anatomical site of interest.

With this version of the system, the targeting key can be configured to have a memory card, receiver and transmission modules, and display all integrated into the key, allowing for simplification of the GLS. The targeting key could also have alternative forms, for example a badge which could be worn by the proceduralist or technologist and could wirelessly and seamlessly transmit the data to the grid.

This version of the device could allow a significant reduction of cost and complexity of the actual grid because the grid simply consists of an array of lighting elements which would be controlled by the targeting key.

In certain embodiments where a targeting key is used, the GLS could simply have lighting fixtures. The GLS can then be configured to illuminate the appropriate lighting fixtures based on the information transmitted from the targeting key.

When used in combination with an MRI system, this version of the system would bypass the need for wireless technology to transmit data through the faraday cage surrounding the MRI scanner.

Typically, an access region of interest is determined based on imaging of the anatomy of a subject, particularly, a real time imaging of a subject. Therefore, in certain embodiments the system disclosed herein comprises an imaging unit configured to image the anatomy of the subject.

An imaging unit can be any suitable imaging unit used to image an anatomy of a subject. An imaging unit can be an x-ray imaging unit, ultrasound imaging unit, magnetic-resonance imaging (MRI) unit, computed tomography (CT) imaging unit, or positron-emission tomography (PET) imaging unit, or PET-CT imaging unit.

Typically, the anatomy of the subject is imaged using an imaging unit to identify the anatomical site of interest. Once the anatomical site of interest is identified, an operator, such as a physician, nurse, or physician's assistant, determines an appropriate access region of interest and appropriate depth of the anatomical site of interest.

In some cases, such information can also be determined by a processor, for example, using artificial intelligence. In such cases, an operator can identify an anatomical site of interest in the imaged anatomy of a subject and the processor can compute the appropriate access region of interest and/or the depth of the anatomical site of interest.

Regardless of how the access region of interest and/or the depth of the anatomical site of interest are determined, such information is provided by the processor to the GLS. Once such information is received by the GLS, the GLS can activate the one or more indicators to identify the access region of interest and the depth of the anatomical site of interest.

For example, as shown in FIG. 10, a processor communicates to the GLS the information about the access region of interest and the GLS indicates the access region of interest by illuminating the LED lights around the access region of interest. The GLS can also further indicate the depth of the anatomical site of interest.

In some cases, the GLS is configured for use in breast biopsies. Typically, the access region of interest in breast biopsies is indicated in two tiered system. First, a larger access region of interest is identified. The larger access region of interest is divided in nine quadrants and second, a specific quadrant from the nine quadrants is identified as the access region of interest. An example of GLS that can be used in breast biopsies is provided in FIG. 9. As seen in FIG. 9, each side of the larger access regions has three LED lights. If, within a larger access region, the far left LED of the horizontal row and the top right LED of the vertical row are illuminated, it indicates that the access region of interest is in the top left quadrant within the larger access region of interest.

One such example is also provided in FIG. 1. The larger access region of interest is the square in the third column and second row. Within this larger access region of interest, the middle LEDs are illuminated indicating that the access region of interest is the middle quadrant of the nine quadrants. Certain details of the access region of interest as used in breast biopsies are given in the Examples 1 and 2 below.

In further embodiments, the system disclosed herein provides automated accessing of the anatomical site of interest. Such automated accessing can be performed by an automated access device (AAD). An ADD can be in communication with the GLS and/or the processor, i.e., the information about the access region of interest and/or the depth of the anatomical site of interest could be transmitted to the AAD by the processor directly or via the GLS.

When actuated, the AAD can access the anatomical site of interest based on the access region of interest and/or the depth of the anatomical site of interest as identified by the GLS and/or the processor.

An AAD can comprise a robot equipped with apparatus for accessing an anatomical site of interest. Apparatus for accessing an anatomical site of interest can comprise a hypodermal needle, vacuum-assisted device, obturator, and sensor. The robotic arm can access the anatomical site of interest based on the access region of interest and/or the depth of the anatomical site of interest as identified by the GLS and/or the processor.

Use of an AAD in combination with the GLS and/or the processor disclosed herein can significantly or fully automate the procedure of accessing an anatomical site of interest. This would increase the speed and accuracy of the procedure while reducing the error and chances of contamination of the anatomical site of interest.

An AAD can be configured to perform any desired procedure at the anatomical site of interest. Non-limiting examples of such procedures include injecting a desired compound, obtaining a biopsy sample, and destroying the cells at the anatomical site of interest, such as cauterizing cells in a cryosurgery.

Any desired compound can be injected at the anatomical site of interest using an AAD. Certain such compounds include an anesthetic, a prophylactic, or a therapeutic compound.

In some cases, an AAD is configured to obtain a biopsy sample from a breast lesion. Certain details of this procedure are described in Examples 1-2 below. AAD can be configured to obtain a biopsy from any desired tissue, which includes placenta, brain, thyroid, parathyroid, thorax, heart, lung, esophagus, thymus, pleura, adrenal glands, appendix, gall bladder, urinary bladder, large intestine, small intestine, kidneys, liver, pancreas, spleen, stoma, prostate, ovaries, uterus, and testis.

### Methods

Further embodiments of the invention provide methods of utilizing the systems disclosed herein to access an anatomical site of interest. Accordingly, certain embodiments of the invention provide a method of accessing an anatomical site of interest in a subject, the method comprising placing the GLS disclosed herein at or near the anatomical site of interest and accessing the anatomical site of interest based on the access region of interest and/or the depth of the anatomical site of interest as identified by the GLS.

Any desired procedure can be performed on the anatomical site of interest. Non-limiting examples of such procedures include injecting a desired compound, obtaining a biopsy sample, and destroying the cells at the anatomical site of interest, such as cauterizing the cells in a cryosurgery.

Any desired compound can be injected at the anatomical site of interest using an AAD. Certain such compounds include an anesthetic, a prophylactic, or a therapeutic compound.

In some cases, a biopsy sample can be obtained from a breast lesion. Certain details of this procedure are described in Examples 1-2 below. A biopsy can be obtained from any desired tissue, which includes placenta, brain, thyroid, parathyroid, thorax, heart, lung, esophagus, thymus, pleura, adrenal glands, appendix, gall bladder, urinary bladder, large intestine, small intestine, kidneys, liver, pancreas, spleen, stoma, prostate, ovaries, uterus, and testis.

The methods disclosed herein typically comprise imaging a subject using an imaging unit and processing the data using a processor and a computer-readable medium comprising instructions that, when executed by the processor, causes the one or more indicators to identify from the plurality of access regions the access region of interest for accessing the anatomical site. Accordingly, an operator can perform imaging of the anatomy of the subject and determine the anatomical site of interest.

An operator can input the chosen anatomical site of interest in a processor and a computer-readable medium comprising instructions that, when executed by the processor, causes the processor to determine from the plurality of access regions the access region of interest for accessing the anatomical site of interest and/or the depth of the anatomical site of interest. The processor and the computer-readable medium can further comprise instructions that, when executed by the processor, causes the processor to transmit to a GLS the information about the access region of interest for accessing the anatomical site of interest and/or the depth of the anatomical site of interest.

Additional details about the system disclosed herein, such as the features of the GLS, types of imaging units, etc. are also applicable to the methods disclosed herein and such embodiments are within the purview of the invention.

### Computer-Readable Media and Devices

Also provided herein are computer readable media for incorporation into the systems disclosed herein and for implementing the methods disclosed herein for accessing an anatomical site of interest.

In certain aspects, provided is a non-transitory computer readable medium including instructions for carrying out the methods disclosed herein, where the instructions, when executed by one or more processors, cause the one or more processors to implement the methods disclosed herein for accessing an anatomical site of interest.

Various steps of accessing an anatomical site of interest may be as described in the Devices and Methods sections above. For purposes of brevity, details regarding these steps and other features/elements described in the Device and Methods sections of the present disclosure are incorporated but not reiterated herein. In some embodiments, the instructions, when executed by one or more processors, cause the one or more processors to perform any of the methods described in the Methods section herein.

Instructions can be coded onto a non-transitory computer-readable medium in the form of “programming,” where the term “computer-readable medium” as used herein refers to any non-transitory storage or transmission medium that participates in providing instructions and/or data to a computer for execution and/or processing. Examples of storage media include a hard disk, optical disk, magneto-optical disk, CD-ROM, CD-R, magnetic tape, non-volatile memory card, ROM, DVD-ROM, Blue-ray disk, solid state disk, network attached storage (NAS), etc., whether such devices are internal or external to the computer. A file containing information can be “stored” on computer readable medium, where “storing” means recording information such that it is later accessible and retrievable by a computer.

The instructions may be in the form of programming that is written in one or more of any number of computer programming languages. Such languages include, for example, Java (Sun Microsystems, Inc., Santa Clara, CA), Visual Basic (Microsoft Corp., Redmond, WA), and C++(AT&T Corp., Bedminster, NJ), as well as many others.

The present disclosure also provides computer devices. The computer devices include one or more processors and any of the non-transitory computer readable media of the present disclosure. Accordingly, in some embodiments, the computer devices can perform any of the methods described in the Methods section herein.

In certain aspects, a computer device of the present disclosure is a local computer device, preferably, a portable computer device, such as a smart-phone or table. In some embodiments, the computer device is a remote computer device (e.g., a remote server), meaning that the instructions are executed on a computer device different from a local computer device and/or the instructions are downloadable from the remote computer device to a local computer device, e.g., for execution on the local computer device. In some embodiments, the instructions constitute a web-based application stored on a remote server.

Notwithstanding the appended claims, the present disclosure is also defined by the following embodiments:

Embodiment 1. A system for localizing an anatomical site of interest in a subject, comprising a grid localization system (GLS), the GLS comprising a plurality of access regions for accessing the subject and one or more indicators configured to identify from the plurality of access regions an access region of interest for accessing the anatomical site of interest.

Embodiment 2. The system of embodiment 1, wherein the GLS further comprises a depth indicator configured to indicate the depth of the anatomical site of interest.

Embodiment 3. The system of embodiment 2, wherein the depth indicator comprises a screen configured to indicate the depth of the anatomical site of interest.

Embodiment 4. The system of any of preceding embodiments, further comprising, in communication with the GLS, a processor and a computer-readable medium comprising instructions that, when executed by the processor, causes the one or more indicators to identify from the plurality of access regions the access region of interest for accessing the anatomical site of interest.

Embodiment 5. The system of any of preceding embodiments, further comprising an imaging unit configured to image the anatomy of the subject.

Embodiment 6. The system of embodiment 5, wherein the imaging unit is an x-ray imaging unit, ultrasound imaging unit, magnetic-resonance imaging (MRI) unit, computed tomography (CT) imaging unit, positron-emission tomography (PET) imaging unit, or PET-CT imaging unit.

Embodiment 7. The system of any of preceding embodiments, wherein the GLS comprises a plurality of quadrangular access regions and wherein the one or more indicators are placed along the sides of the quadrangular access regions.

Embodiment 8. The system of any of preceding embodiments, wherein the one or more indicators comprise visual indicators.

Embodiment 9. The system of embodiment 8, wherein the visual indicators comprise light elements.

Embodiment 10. The system of embodiment 9, wherein the light elements comprise light-emitting diodes.

Embodiment 11. The system of any of preceding embodiments, wherein the processor is in communication with the GLS via a wired connection.

Embodiment 12. The system of embodiment 11, wherein the processor is in communication with the GLS via a wireless connection.

Embodiment 13. The system of embodiment 12, wherein the wireless connection is via Bluetooth, Wi-Fi, Wi-MAX, or cellular network.

Embodiment 14. The system of any of preceding embodiments, wherein the GLS comprises an adhesive on the side opposite to the side comprising the one or more indicators.

Embodiment 15. The system of embodiment 14, wherein the adhesive is a skin compatible adhesive.

Embodiment 16. The system of any of preceding embodiments, further comprising an automated access device (AAD) that is in communication with the GLS and/or the processor, wherein the AAD, when actuated, accesses the anatomical site of interest based on the access region of interest and/or the depth of the anatomical site of interest as identified by the GLS and/or the processor.

Embodiment 17. The system of embodiment 16, wherein the AAD comprises one or more of: a hypodermal needle, vacuum-assisted device, obturator, and sensor.

Embodiment 18. The system of embodiment 16 or 17, wherein the AAD, when actuated, obtains a biopsy sample from the anatomical site of interest.

Embodiment 19. The system of embodiment 18, wherein the AAD, when actuated, obtains a breast or a prostate biopsy sample from the anatomical site of interest.

Embodiment 20. The system of embodiment 16 or 17, wherein the AAD, when actuated, injects a compound to the anatomical site of interest.

Embodiment 21. A method of accessing an anatomical site of interest of a subject, the method comprising placing the GLS of any of embodiments 1 to 15 at or near the anatomical site of interest of the subject and accessing the anatomical site of interest based on the access region of interest and/or the depth of the anatomical site of interest as identified by the GLS.

Embodiment 22. The method of embodiment 21, wherein accessing the anatomical site of interest comprises obtaining a biopsy sample from the anatomical site of interest.

Embodiment 23. The method of embodiment 22, comprising obtaining a breast or a prostate biopsy sample.

Embodiment 24. The method of embodiment 21, wherein accessing the anatomical site of interest comprises injecting a compound to the anatomical site of interest.

Embodiment 25. A system comprising a processor and a computer-readable medium comprising instructions that, when executed by the processor, causes the processor to determine from a plurality of access regions in a GLS of any of embodiments 1-15, the access region of interest for accessing an anatomical site of interest and/or a depth of the anatomical site of interest.

Embodiment 26. The system of embodiment 25, wherein the computer-readable medium further comprises instructions, that when executed by the processor, causes the processor to transmit to the GLS the information about the access region of interest for accessing the anatomical site of interest and/or the depth of the anatomical site of interest.

Embodiment 27. The system of embodiment 25 or 26, further comprising an imaging unit configured to image the anatomy of a subject.

Embodiment 28. The system of embodiment 27, wherein the imaging unit is an x-ray imaging unit, ultrasound imaging unit, magnetic-resonance imaging (MRI) unit, computed tomography (CT) imaging unit, positron-emission tomography (PET) imaging unit, or PET-CT imaging unit.

### EXPERIMENTAL

### Example 1—the Devices and Methods of the Invention Used to Obtain Breast Biopsies

Certain embodiments of the invention provide GLS that guides the localization of a biopsy site in a subject. For example, the GLS can be an MRI-Biopsy Grid Localization System (MGLS) that facilitates the localization of a biopsy site in a subject in an MRI-guided biopsy procedure.

The MGLS comprises a plastic open-grid with an integrated matrix of light emitting diodes (LED) that correspond to Cartesian coordinates used for lesion localization at the time of biopsy. This plastic grid can either replace the existing commercially available standard MRI-biopsy grids or can be affixed to the existing grids (FIG. 1). The grid can contain a power supply, switching regulator, microcontroller, capacitors, transistors, resistors, circuit board, integrated circuits and series of LED lighting elements imbedded within the grid. The grid can also have an integrated Bluetooth receiver module and LCD/TFT display with display module to display additional procedural data. For the breast biopsy system, each square grid space can have 12 lighting elements (3 elements per side). Each grid space accommodates a biopsy plug with nine available biopsy ports. Each plug position would be represented by two illuminated LED lights, with one LED illuminated for the X-axis and one LED illuminated for the Y-axis. A center plug location could be represented by the entire grid illuminated (all 9 lighting elements) or a stellate configuration (two center x-axis LEDS and two center Y-axis LEDs). Center plug position could also be displayed on the LCD/TFT display.

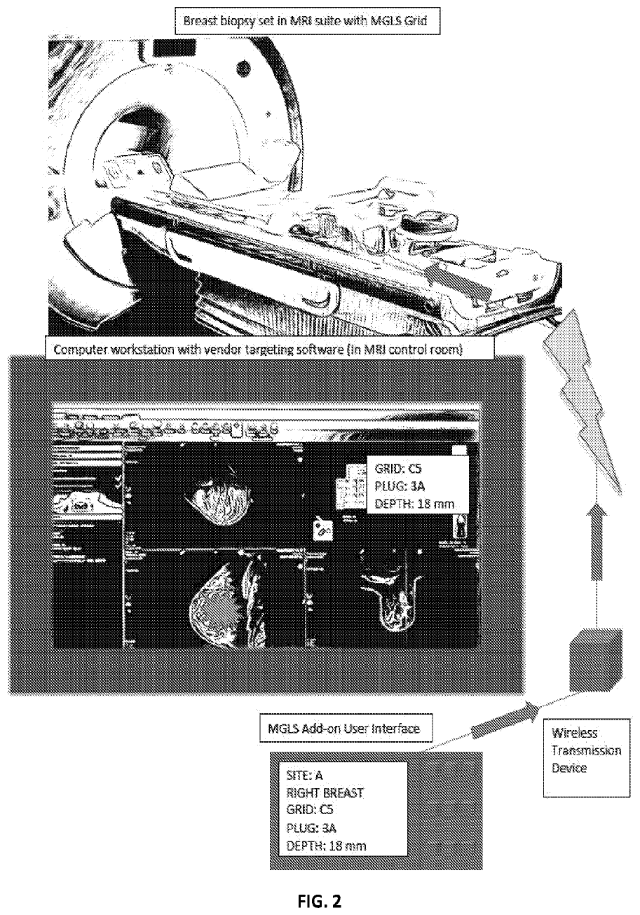

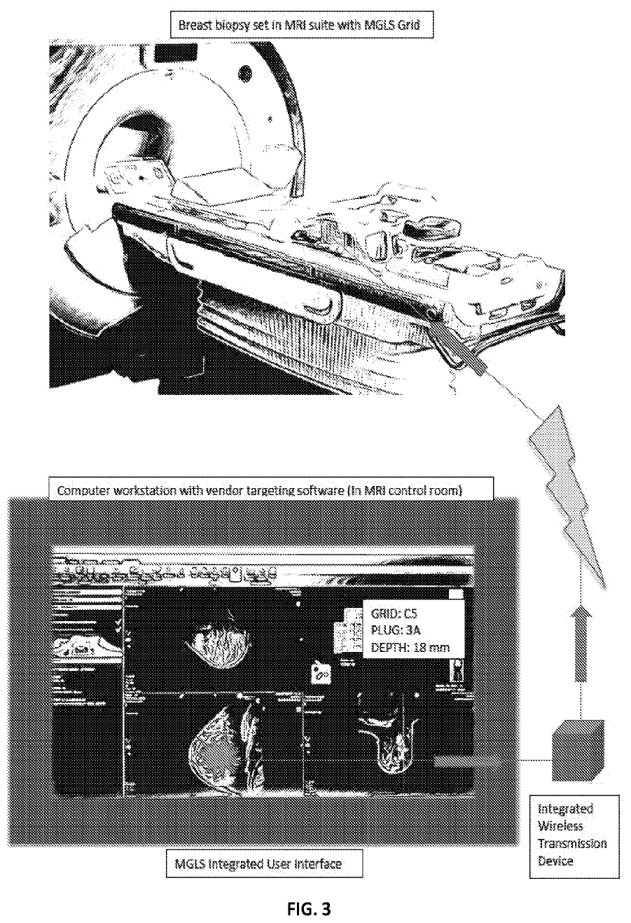

Following patient positioning and the acquisition of dynamic contrast-enhanced images, the proceduralist performs the lesion targeting utilizing standard vendor-specific software at the computer workstation in the MRI-control room. The MGLS with the add-on user interface includes an LCD/TFT display, display module, keypad and radiofrequency (RF) transmission module. This system contains a power supply, switching regulators, microcontrollers, capacitors, transistors, resistors, circuit board, and integrated circuits that can allow the proceduralist to input lesion location (lesion, grid, plug position, depth) and then instantaneously transmit the biopsy or localization coordinates from the MRI control room to the biopsy grid located in the MRI suite, where the procedure is performed (FIG. 2). Alternatively, a software patch could allow for integrated targeting and transmission of the biopsy data to the grid directly from the workstation utilizing an integrated transmission system (FIG. 3). In this case, a simple transmitter containing a radiofrequency transmission module can be connected to computer workstation allowing for integrated targeting and data transmission utilizing vendor software. A shielded backup cable would also be used to allow for transmission of biopsy data from the workstation to the grid.

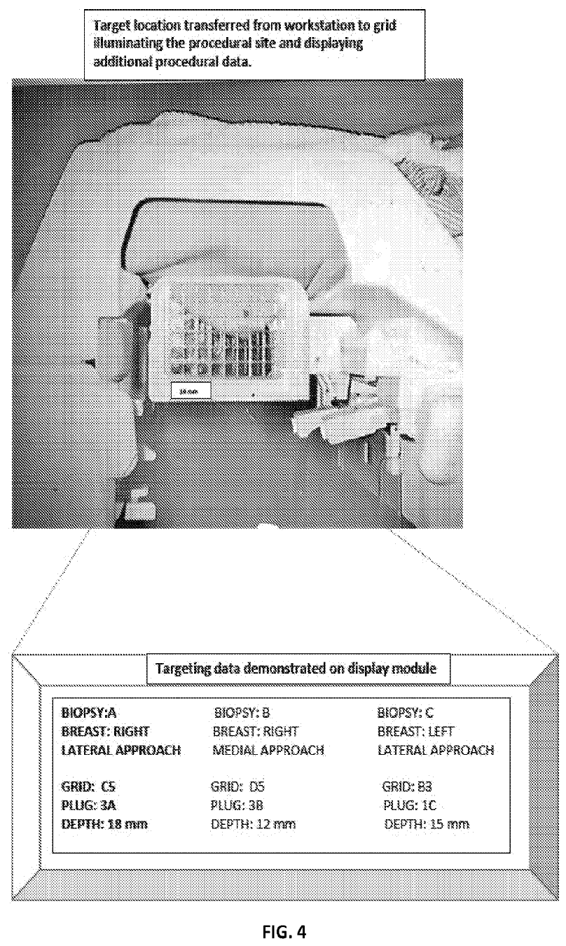

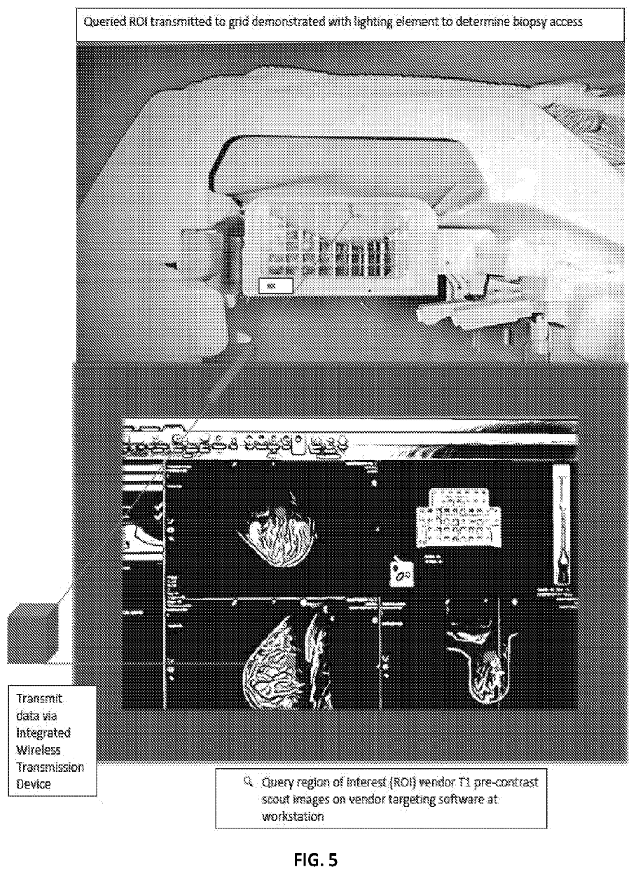

The grid receives the biopsy/localization coordinates via the integrated receiver module and is configured to illuminate light-emitting diodes within the grid located at the correct procedural site(s) for biopsy as specified by the targeting software (FIG. 4). Additional procedural information such as target lesion depth, biopsy approach, and multiple site targeting data can be presented on the integrated grid display (FIG. 5).

In addition to streamlining and integrating procedural localization, the MGLS offers additional tools to aid in both positioning and pre-procedural planning. Utilizing an integrated software patch for the vendor targeting software, the proceduralist can draw a region-of-interest (ROI) over the suspected region where the lesion is located on the pre-contrast scout images (FIG. 5). The volumetric area corresponding to the queried ROI can be processed via proprietary software and transmitted to the grid utilizing the integrated transmission system. The receiver module in the grid would receive the volumetric data and would illuminate light-emitting diodes within the grid corresponding to the queried ROI (FIG. 5). This program would allow for quick visual evaluation of the patient and confirmation of access to the available biopsy sites, allowing for optimization of patient positioning prior to the administration of intravenous contrast. This process would be very helpful for small lesions (foci) or for lesions close to critical structures like the nipple-areolar complex or chest wall.

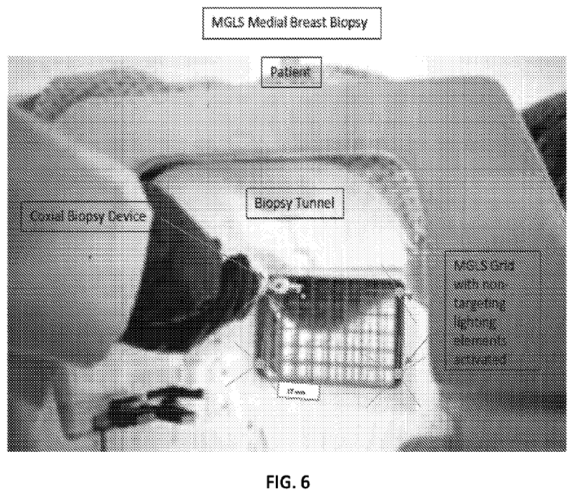

The MGLS can have numerous integrated lighting elements, with additional elements that could be used illuminate the dark biopsy tunnel during medial approach breast biopsies. These lighting elements aid the proceduralist by allowing for easy visualization of both the biopsy site and coaxial biopsy system (FIG. 6).

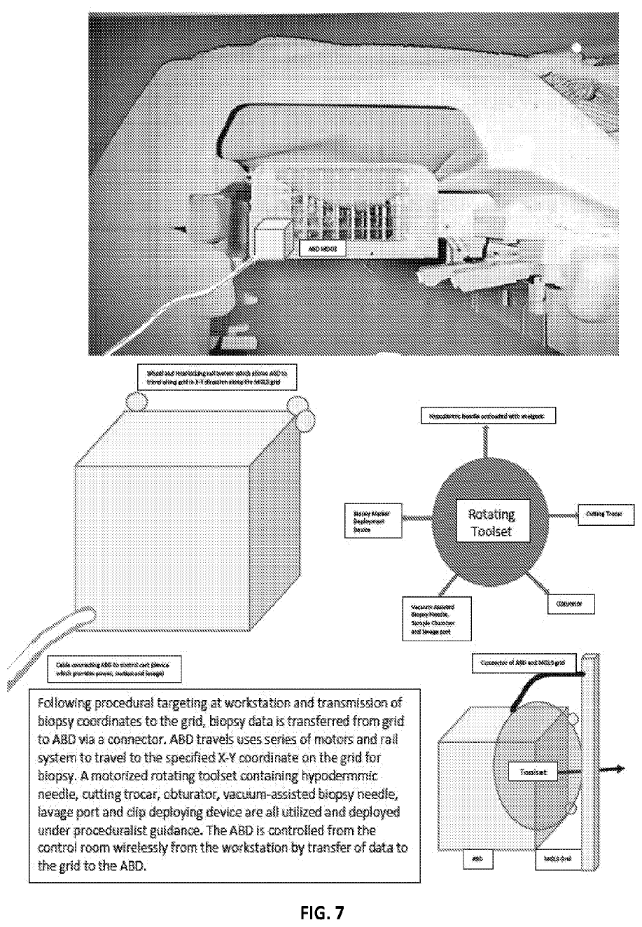

The MGLS transmission and receiver system can be used in conjunction with an automated biopsy device (ABD) system. A software patch allows integrated targeting and transmission of the biopsy targeting data to the grid from the workstation utilizing the transmission system. The grid receives the biopsy coordinates via the receiver module and transmits the biopsy coordinates to an ABD (FIG. 7). In lieu of a matrix of light emitting diodes, the grid can contain a rail-system that allows ABD to travel on the grid to the biopsy site as specified by the targeting software (FIG. 7). To perform various functions required to obtain the biopsy, the ABD can have integrated tools, such as hypodermal needle supplied with lidocaine, vacuum-assisted device system, obturator, computer module, and sensors (FIG. 13).

As such, in the GLS disclosed herein, biopsy target data is transmitted to a grid system placed on or near the biopsy site to facilitate biopsy. GLS facilitates quicker biopsies, reduces human error, and improves preprocedural planning. In certain embodiments, the GLS further comprises an automated biopsy device (ABD), which, based on the biopsy target data transmitted from the GLS, can perform the biopsy in an automated manner, i.e., without significant human intervention in the physical step of taking a biopsy from the subject. Further embodiments of the invention provide methods of taking a biopsy sample from a subject by implementing the GLS and/or ABD disclosed herein.

The devices and methods disclosed herein offers additional tools to improve patient positioning and pre-procedural planning in MRI-guided breast biopsies. For MRI-guided breast biopsies, the breast is placed in compression and TI weighted pre-contrast scout images are acquired under the standard procedural protocol, with the objective of confirming that area of the suspected lesion is safely accessible within the grid. The proceduralist utilizes anatomical landmarks, such as the fat-glandular tissue interface or other lesions as a guide to approximate the expected location of the lesion prior to the administration of intravenous contrast medium. Lesions can sometimes be small, and changes of breast anatomy during compression can make the approximation challenging. Once the patient receives the contrast bolus, the proceduralist is committed to the current patient positioning for the duration of the procedure, with the result sometimes being an inaccessible biopsy site secondary to the target being located outside available grid spaces or located too close to vital structure such as the nipple or chest wall. With the region-of-interest (ROI) query tool of the MRI-Biopsy Grid Localization System (MGLS), the proceduralist would have the ability to draw a region-of-interest (ROI) over the suspected area where the target lesion is located at the workstation on the pre-contrast scout images utilizing vendor targeting software. The volumetric area corresponding to the queried ROI would be transmitted to the grid and would illuminate the corresponding light-emitting diodes within the grid. This tool allows the proceduralist to quickly evaluate the patient and confirm access to the available biopsy sites, allowing for changes in patient positioning to optimize lesion accessibility prior to the administration of intravenous contrast.

The use of imbedded lighting elements in the offer several advantages, including the ability to illuminate of the grid and coaxial biopsy system during medial breast biopsies. Medial breast biopsies, which are performed within a dark tunnel underneath the patient make the visualization of the procedural site and coaxial system challenging due to limited lighting. The proceduralist often needs extra lighting, a task currently facilitated by a nurse/MRI technologist holding a flashlight.

Finally, an automated biopsy system disclosed herein can be implemented to obtain breast biopsies. In such embodiments, the target information received by the grid can be transferred to the automated systems which could use the biopsy coordinates to perform the biopsy autonomously.

### Example 2—MGLS Provides Superior Results Compared to Conventional Methods

Compared to the conventional methods, MGLS demonstrates improvements in multiple parameters including:
1) Reduced overall biopsy time for single site biopsy and cost savings from decreased MRI scanner utilization.
2) Reduced biopsy time for multiple site biopsies and cost savings from decreased MRI scanner utilization.
3) Increased number of successful biopsies.
4) Reduced errors.

MRI biopsy/localization procedures are technically challenging, with lesions often not visible on other imaging modalities. Challenges encountered with patient anatomy, positioning, and contrast enhancement all come into play during the MRI-guided procedures. MGLS disclosed herein offers several advantages over the conventional localization grid, including improvements in procedural efficiency and pre-procedural planning, reductions in human error, improved accessibility for medial breast biopsies, and the availability of implementing automated biopsy systems. Given the inherent complexities and challenges of MRI-guided procedures, the devices and methods disclosed herein allow for process simplification, decreased cost, and improved patient outcomes.

The preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A system for localizing an anatomical site of interest in a subject, comprising a grid localization system (GLS), the GLS comprising a plurality of access regions for accessing the subject and one or more indicators configured to identify from the plurality of access regions an access region of interest for accessing the anatomical site of interest.

2. The system of claim 1, wherein the GLS further comprises a depth indicator configured to indicate the depth of the anatomical site of interest.

3. The system of claim 2, wherein the depth indicator comprises a screen configured to indicate the depth of the anatomical site of interest.

4. The system of claim 1, further comprising, in communication with the GLS, a processor and a computer-readable medium comprising instructions that, when executed by the processor, causes the one or more indicators to identify from the plurality of access regions the access region of interest for accessing the anatomical site of interest.

5. The system of claim 1, further comprising an imaging unit configured to image the anatomy of the subject.

6. The system of claim 5, wherein the imaging unit is an x-ray imaging unit, ultrasound imaging unit, magnetic-resonance imaging (MRI) unit, computed tomography (CT) imaging unit, positron-emission tomography (PET) imaging unit, or PET-CT imaging unit.

7. The system of claim 1, wherein the GLS comprises a plurality of quadrangular access regions and wherein the one or more indicators are placed along the sides of the quadrangular access regions.

8. The system of claim 1, wherein the one or more indicators comprise visual indicators.

9. The system of claim 8, wherein the visual indicators comprise light elements.

10. (canceled)

11. The system of claim 1, wherein the processor is in communication with the GLS via a wired connection or wireless connection.

12. (canceled)

13. (canceled)

14. The system of claim 1, wherein the GLS comprises an adhesive on the side opposite to the side comprising the one or more indicators.

15. (canceled)

16. The system of claim 1, further comprising an automated access device (AAD) that is in communication with the GLS and/or the processor, wherein the AAD, when actuated, accesses the anatomical site of interest based on the access region of interest and/or the depth of the anatomical site of interest as identified by the GLS and/or the processor.

17. The system of claim 16, wherein the AAD comprises one or more of: a hypodermal needle, vacuum-assisted device, obturator, and sensor.

18. The system of claim 16, wherein the AAD, when actuated, obtains a biopsy sample from the anatomical site of interest.

19. The system of claim 18, wherein the AAD, when actuated, obtains a breast or a prostate biopsy sample from the anatomical site of interest.

20. The system of claim 16, wherein the AAD, when actuated, injects a compound to the anatomical site of interest.

21. A method of accessing an anatomical site of interest of a subject, the method comprising placing the GLS of claim 1 at or near the anatomical site of interest of the subject and accessing the anatomical site of interest based on the access region of interest and/or the depth of the anatomical site of interest as identified by the GLS.

22. (canceled)

23. (canceled)

24. (canceled)

25. A system comprising a processor and a computer-readable medium comprising instructions that, when executed by the processor, causes the processor to determine from a plurality of access regions in a GLS of claim 1, the access region of interest for accessing an anatomical site of interest and/or a depth of the anatomical site of interest.

26. The system of claim 25, wherein the computer-readable medium further comprises instructions, that when executed by the processor, causes the processor to transmit to the GLS the information about the access region of interest for accessing the anatomical site of interest and/or the depth of the anatomical site of interest.

27. (canceled)

28. The system of claim 26, wherein the imaging unit is an x-ray imaging unit, ultrasound imaging unit, magnetic-resonance imaging (MRI) unit, computed tomography (CT) imaging unit, positron-emission tomography (PET) imaging unit, or PET-CT imaging unit.
